(19) United States
(12) Patent Application Publication
(10) Pub. No.: US 2007/0179490 A1
(43) Pub. Date: Aug. 2, 2007

(54) METHODS, DEVICES AND SYSTEMS FOR HAIR REMOVAL

(76) Inventors: Zion Azar, Shoham (IL); Pinchas Shalev, Herzlia (IL)

Correspondence Address: DANIEL J SWIRSKY, 55 REUVEN ST., BEIT SHEMESH, 99544, omitted

(21) Appl. No.: 11/669,960

(22) Filed: Feb. 1, 2007

Related U.S. Application Data
(60) Provisional application No. 60/763,898, filed on Feb. 1, 2006.

(51) Int. Cl. A61B 18/04; A61B 18/18

(52) U.S. Cl. 606/28; 606/36; 606/43

(57) ABSTRACT

Methods, Devices and systems for hair removal combine the application of RF electromagnetic energy to the skin with application of heat from heating element(s) to hair(s) of the skin. The devices or systems may include RF electrodes and one or more heating elements. RF energy from an RF generating unit is applied to the RF electrodes. Heat is applied to hair shafts by the heating element(s). The devices and systems may be hand held or may include a hand held part connected to a base unit. In operation, the hand held device or part is moved along the treated skin region. The combined effect of RF energy application to the skin and heating of the hair shaft results in improved hair removal and may reduce hair re-growth. The devices and systems may also include safety improving features preventing skin damage by reducing sparking or excessive skin heating by the heating element(s).

### CROSS-REFERENCE TO RELATED US APPLICATIONS

This application claims priority from and the benefit of U.S. Provisional Patent Application Ser. No. 60/763,898 filed Feb. 1, 2006, entitled “METHODS AND DEVICES FOR HAIR REMOVAL”, incorporated herein by reference in its entirety.

### FIELD OF THE INVENTION

The present invention relates to devices, systems and methods for removing facial and body hair and more specifically to devices systems and methods for removing hair using a combination of heating element(s) and RF energy application.

### BACKGROUND OF THE INVENTION

The removal of unwanted facial and body hair can be accomplished with non-mechanized means, such as, for example, razors, tweezers or wax, all of which are uncomfortable to use, irritate the skin and/or cause damage to the skin.

Mechanized cutting means for cutting hair, for example dry shavers, in addition to being uncomfortable to use, are limited to cutting hair of a specific length. Beard trimmers, for example, cut facial hair stubble, but cannot cut longer hairs on the scalp.

The use of heated wires or other structures to cut hair from a skin surface has been proposed. However, a heat generator that generates heat of a sufficient magnitude to cut hair and that cuts the hair close to the skin, often damages the skin. Alternatively, since the heat generator is offset from the skin to prevent skin damage, unwanted stubble is left behind.

U.S. Pat. No. 3,934,115 to Peterson, discloses the use of parallel metal strips on the upper side of a ceramic facing that contacts the skin, are used to cut hair. U.S. Pat. No. 2,727,132 to Hills and Italian Patent IT 1201364P to Massimo disclose a continuously heated element for burning hair. U.S. Pat. No. 558,465, to Bell and U.S. Pat. No. 589,445, to D. Seide, U.S. Pat. No. 2,727,132 to G. S. Hills, U.S. Pat. No. 3,093,724 to G. L. Johnson, U.S. Pat. No. 5,064,993 to Hashimoto and U.S. Pat. No. 6,307,181 to Hashimoto, French Patent FR 2531655 to F. Solvinto, European Patent EP 0201189 to F. Solvinto, and French Patent No. FR 2612381 to E. Michit, all disclose a continuously heated wire for burning hair. U.S. Pat. No. 3,474,224 to J. F. Carter, discloses a circular comb device for burning nose hairs. Aside from physically separating the skin from the heated element, the above cited references do not appear to provide other protection against burning of the skin.

All the above devices and methods are in use for short term hair removal and do not provide permanent hair reduction.

Other types of devices are directed to long term hair removal. Electrolysis devices are based on the use of “electric needles”. Such fine needles are inserted into the hair follicle and apply an electric current to each hair. The current heats the hair and causes its carbonization and also heats the tissue near the hair causing its coagulation and partial or full coagulation of the blood capillaries which supply blood to the hair follicle. While such devices can result in permanent hair removal, each hair must be treated individually, making hair removal by this method a tedious often painful, time consuming, and expensive.

Another class of known devices includes photothermolysis devices which are usually operated by physicians in clinics. These devices are based either on lasers (e.g. Ruby lasers) such as the laser device disclosed in U.S. Pat. No. 5,059,192 to Zaias or on an incoherent light source coupled with filters and elaborate electronics to provide pulses of various durations and wave lengths as described in U.S. Pat. No. 5,405,368 to Eckhouse and in European Patent publications EP 0 788 814 and EP 0736308. The above referenced Eckhouse patents and European Publications disclose heating the hair directly by a high flux of visible radiation that is absorbed by the hair follicles. Various filters and/or pulse lengths are used depending on the depth of penetration desired and the color of the hair being removed.

Another type of long term hair removal method, disclosed in U.S. Pat. No. 6,702,808 is based on using RF energy coupled with a light energy that provides selective pre-heating of the hair to be destroyed. Light energy is applied to the skin and selectively pre-heats the hair follicle above the temperature of the surrounding skin but below the coagulation temperature, RF energy is then applied to the skin to cause coagulation of the hair follicle since the applied RF energy causes more heating of the heated hair follicle than the surrounding skin. In order to achieve selective pre-heating of the hair follicle, the color of the hair should be darker than the surrounding skin. Thus this method suffers from the same deficiencies as Photothermolysis devices.

There is therefore a need for device and method for short long term hair removal or hair reducing that do not depends on the contrast between the color of the skin and the color of the hair.

### SUMMARY OF THE INVENTION

There is therefore provided a device for hair removal. The device includes a housing, at least one RF electrode attached to the housing for applying RF electrical currents to the skin for heating at least part of the skin and at least one heating unit configured for applying heat to at least a portion of at least one hair of the skin.

Furthermore, in accordance with an embodiment of the device, the at least one RF electrode attached to the housing includes an active electrode which forms part of a unipolar RF electrode arrangement. The unipolar RF electrode arrangement also includes a return RF electrode attachable to a distant part of the skin.

Furthermore, in accordance with an embodiment of the device, the device includes two RF electrodes arranged in a bipolar electrode configuration.

Furthermore, in accordance with an embodiment of the device, the device includes a plurality of RF electrodes attached to the housing.

Furthermore, in accordance with an embodiment of the device, the at least one RF electrode is (or are) electrically connectable to an RF current generating unit.

Furthermore, in accordance with an embodiment of the device, the RF current generating unit is selected from an RF current generating unit disposed within the housing and an RF current generating unit disposed outside of the housing.

Furthermore, in accordance with an embodiment of the device, the RF current generating unit has a nominal RF power rating in the range of 1-20 watt. However, RF power ratings higher than 20 watt or lower than 1 watt may also be used in accordance with other embodiments of the device.

Furthermore, in accordance with an embodiment of the device, the RF current generating unit is configured for providing RF energy to the skin in an energy delivery mode selected from pulsed RF, continuous wave RF and quasi-continuous wave RF.

Furthermore, in accordance with an embodiment of the device, the RF current generating unit is configured for providing RF voltages having an RF frequency range of 200 KHz-100 MHz.

Furthermore, in accordance with an embodiment of the device, the RF current generating unit is an RF current generating unit disposed within the housing and the device also includes an internal electrical power source for energizing the heating unit(s) and the RF current generating unit.

Furthermore, in accordance with an embodiment of the device, the internal electrical power source may be a direct current power supply, an alternating current power supply, a fuel cell, a battery, a primary electrochemical cell or a rechargeable electrochemical cell.

Furthermore, in accordance with an embodiment of the device, the RF current generating unit is an RF current generating unit disposed outside the housing and the at least one heating unit and the RF current generating unit are energized by an electrical power source disposed outside the device.

Furthermore, in accordance with an embodiment of the device, the at least one heating unit is selected from a fixed heating unit, a movable heating unit, a removable heating unit, a detachable heating unit, a replaceable heating unit, a disposable heating unit and combinations thereof.

Furthermore, in accordance with an embodiment of the device, the at least one heating unit includes one or more heating elements.

Furthermore, in accordance with an embodiment of the device, the heating element(s) are selected from one or more electrically resistive wires, one or more metallic wires, one or more metallic ribbons, one or more ceramic heating elements, one or more metalized ceramic elements and combinations thereof.

Furthermore, in accordance with an embodiment of the device, the device is configured as a hand held device connectable to a base station.

Furthermore, in accordance with an embodiment of the device, the base station comprises an RF current generating unit for energizing the at least one RF electrode.

Furthermore, in accordance with an embodiment of the device, the base station also includes an electrical power source for energizing the heating unit(s).

Furthermore, in accordance with an embodiment of the device, the base station includes a controller unit for controlling the application of RF energy to the at least one RF electrode.

Furthermore, in accordance with an embodiment of the device, the base station includes a controller unit for controlling the heating of said heating unit.

Furthermore, in accordance with an embodiment of the device, the hand-held device includes one or more sensor units and the base station includes circuitry for receiving signals from the sensor unit(s) and for processing the signals.

Furthermore, in accordance with an embodiment of the device, the at least one RF electrode is at least one active RF electrode, the base station also includes at least one return RF electrode, and the sensor unit(s) include sensor(s) for determining the impedance between the at least one active RF electrode and the at least one return RF electrode.

Furthermore, in accordance with an embodiment of the device, the sensor unit(s) include sensors for providing signals representative of the velocity of the hand-held device relative to the skin.

Furthermore, in accordance with an embodiment of the device, the at least one RF electrode includes at least two RF electrodes arranged in a bipolar configuration. The device also includes an RF current generating unit controllably connectable to the at least two RF electrodes. The device also includes an impedance determining unit for determining the electrical impedance between the two RF electrodes. The device also includes a controller unit coupled to the RF current generating unit and to the impedance determining unit. The controller unit is configured for terminating the application of RF currents to the at least two RF electrodes when the impedance value between the at least two RF electrodes exceeds an impedance threshold value and for initiating the application of RF currents to the at least two RF electrodes when the impedance value between the at least two RF electrodes is equal to or smaller than the impedance threshold value.

Furthermore, in accordance with an embodiment of the device, the at least one RF electrode includes at least one actively cooled RF electrode.

Furthermore, in accordance with an embodiment of the device, the at least one actively cooled RF electrode is selected from a hollow RF electrode having at least one passage therewithin for coolant flow within said RF electrode, and an RF electrode including a Peltier element.

Furthermore, in accordance with an embodiment of the device, the at least one RF electrode includes an RF electrode having at least one porous portion capable of containing an electrically conducting solution therewithin.

There is also provided a system for hair removal. The system includes RF electrodes for applying RF electrical currents to the skin, an RF current generating unit controllably connectable to the RF electrodes, and at least one heating unit for applying heat to at least a portion of at least one hair of the skin.

Furthermore, in accordance with an embodiment of the system, the system includes a controller unit for controlling the application of the RF electrical currents to the skin.

Furthermore, in accordance with an embodiment of the system, the controller unit also controls the operation of the heating unit.

Furthermore, in accordance with an embodiment of the system, the system includes a controller unit for controlling the operation of the heating unit.

Furthermore, in accordance with an embodiment of the system, the system includes an impedance determining unit for determining the value of the electrical impedance between the RF electrodes. The impedance determining unit is connected to the controller and to the RF electrodes. The controller is configured for terminating the application of RF currents to the skin when the value of the impedance between the RF electrodes exceeds a threshold impedance value.

Furthermore, in accordance with an embodiment of the system, the system also includes a speed determining unit for determining the velocity of movement of the RF electrodes along the skin.

Furthermore, in accordance with an embodiment of the system, the speed determining unit is connected to at least one sensor.

Furthermore, in accordance with an embodiment of the system, the at least one sensor is mechanically coupled to at least one of the RF electrodes.

Furthermore, in accordance with an embodiment of the system, the system includes a hand held unit including one or more of the RF electrodes and the heating unit. The system also includes base unit connected to the hand held unit. The base unit includes at least the RF generating unit.

Furthermore, in accordance with an embodiment of the system, the base unit also includes a processor/controller unit for controlling the operation of the RF generating unit.

Furthermore, in accordance with an embodiment of the system, the base unit also includes an impedance determining unit for determining the value of the electrical impedance between the RF electrodes. The impedance determining unit is connected to the processor/controller unit and to the RF electrodes. The processor/controller unit is configured for terminating the application of RF currents to the skin when the value of the impedance between the RF electrodes exceeds a threshold impedance value.

Furthermore, in accordance with an embodiment of the system, the base unit also includes a speed determining unit connected to one or more sensors included in the hand held unit for determining the velocity of movement of the hand held unit along the skin. The heating unit is a movable heating unit and the processor/controller unit is configured for terminating the heating of the heating unit if the value of the velocity of movement is less then a threshold value.

Furthermore, in accordance with an embodiment of the system, the RF current generating unit has a nominal RF power rating in the range of 1-20 watt.

Furthermore, in accordance with an embodiment of the system, the RF current generating unit is configured for providing RF energy to the skin in an energy delivery mode selected from pulsed RF, continuous wave RF and quasi-continuous wave RF.

Furthermore, in accordance with an embodiment of the system, the RF current generating unit is configured for providing RF voltages having an RF frequency range of 200 KHz-100 MHz.

There is also provided a method for hair removal, the method includes the steps of applying to the skin RF electromagnetic energy and applying heat from a heating element to at least one portion of at least one hair of the skin.

Furthermore, in accordance with an embodiment of the method, the step of applying heat from a heating element includes heating said at least one portion sufficiently to induce a tissue coagulation wave propagating along at least a portion of the tissues surrounding the at least one hair.

Furthermore, in accordance with an embodiment of the method, the step of applying heat from a heating element includes heating the at least one portion of the hair sufficiently to cut the at least one hair.

Furthermore, in accordance with an embodiment of the method, the step of applying heat from a heating element includes heating the at least one portion sufficiently to ignite the at least one hair.

Furthermore, in accordance with an embodiment of the method, the method also includes the steps of moving the heating element along the skin, determining the velocity of movement of the heating element relative to the skin, and terminating the heating of the heating element if the value of the velocity of movement is less then a threshold value.

Furthermore, in accordance with an embodiment of the method, the method also includes the step of initiating the heating of the heating element if the value of the velocity of movement is larger than the threshold value.

Furthermore, in accordance with an embodiment of the method, the method also includes the step of moving the heating element along the skin, determining the velocity of movement of the heating element relative to the skin, and increasing the distance between the heating element and the skin if the value of the velocity of movement along said skin is smaller then a threshold value.

Furthermore, in accordance with an embodiment of the method, the method also includes the step of decreasing the distance between the heating element and the skin if the value of the velocity of movement along said skin is larger than the threshold value.

Furthermore, in accordance with an embodiment of the method, the step of applying to the skin RF electromagnetic energy includes the step of passing RF currents through the skin by applying RF voltage to the skin.

Furthermore, in accordance with an embodiment of the method, the RF electromagnetic energy has a nominal RF power in the range of 1-20 watt.

Furthermore, in accordance with an embodiment of the method, the RF electromagnetic energy is applied in an energy delivery mode selected from pulsed RF, continuous wave RF and quasi-continuous wave RF.

Furthermore, in accordance with an embodiment of the method, the RF electromagnetic energy has an RF frequency range of 200 KHz-100 MHz.

Furthermore, in accordance with an embodiment of the method, the RF voltage is a pulsed RF voltage having a pulse repetition rate in the range of 100 Hz-25 KHz and an RF pulse duration in the range of 0.01-2 milliseconds.

Furthermore, in accordance with an embodiment of the method, the step of applying to the skin RF electromagnetic energy also includes the steps of determining the value of the impedance between at least two RF electrodes used for applying the RF electromagnetic energy to said skin, and terminating the applying of RF electromagnetic energy to the skin if the value of the impedance exceeds a threshold value.

Furthermore, in accordance with an embodiment of the method, the step of applying to the skin RF electromagnetic energy also includes the step of initiating the applying of RF electromagnetic energy to the skin if the value of the impedance is smaller than the threshold value.

There is also provided a method for hair removal. The method includes the steps of subjecting the skin to RF electromagnetic radiation, and applying heat to a portion of at least one hair of the skin to reduce the resistance of at least a portion of tissue adjacent to the at least one hair to flow of RF currents therethrough to form in skin tissues adjacent the at least one hair a moving coagulation zone advancing in the skin tissues to at least partially coagulate a portion of the skin tissues.

Finally, there is also provided a method for hair removal. The method includes the steps of applying to the skin RF electromagnetic energy insufficient to cause tissue coagulation of said skin by itself and applying heat to at least one portion of at least one hair of the skin. The applying ignites the at least one hair and produces an RF current induced tissue coagulation wave propagating along at least a portion of the tissues surrounding the at least one hair.

### BRIEF DESCRIPTION OF THE DRAWINGS

The invention is herein described, by way of example only, with reference to the accompanying drawings, in which like components are designated by like reference numerals, wherein:

FIG. 1 is a schematic block diagram illustrating the components of a device for hair removal in accordance with an embodiment of the present invention;

FIG. 2 is a schematic block diagram illustrating the components of another device for hair removal in accordance with an embodiment of the present invention;

FIGS. 3-5 are schematic cross-sectional diagrams illustrating different successive stages of a skin region during treatment by a hair removal device of the present invention having a single heating element and a bipolar RF electrode arrangement, in accordance with an embodiment of the present invention;

FIG. 6 is a is a schematic isometric view illustrating a hair removal system including a base station and a hand held part, in accordance with an embodiment of the present invention;

FIG. 7 is a schematic isometric view of a hand held hair removing device, in accordance with another embodiment of the present invention;

FIG. 8 is a schematic diagram illustrating a system for hair removal including various safety features, in accordance with an embodiment of the present invention.

FIG. 9 is a schematic top view illustrating an experimental device for demonstrating and testing the heat induced RF coagulation cascade effect used in the present invention; and

FIG. 10 is a schematic isometric view illustrating a battery operated hand held hair removing device having a movable heating unit, in accordance with another embodiment of the present invention.

### DETAILED DESCRIPTION OF THE INVENTION

### Notation Used Throughout

### The following notation is used throughout this document.

| Term | Definition |
|---|---|
| A | Ampere |
| CW | Continuous wave |
| Hz | Hertz |
| KHz | Kilohertz |
| mA | Milliampere |
| MHz | Megahertz |
| msec | Millisecond |
| Quasi-CW | Quasi-continuous wave |
| RF | Radio Frequency |
| W | Watt |

The invention described in the present application includes, inter alia, methods, devices and systems for hair removal using a combination of a heat source (such as, for example, one or more heating elements) for applying heat to hairs and an RF (Radio Frequency) source for applying radio frequency currents to the skin.

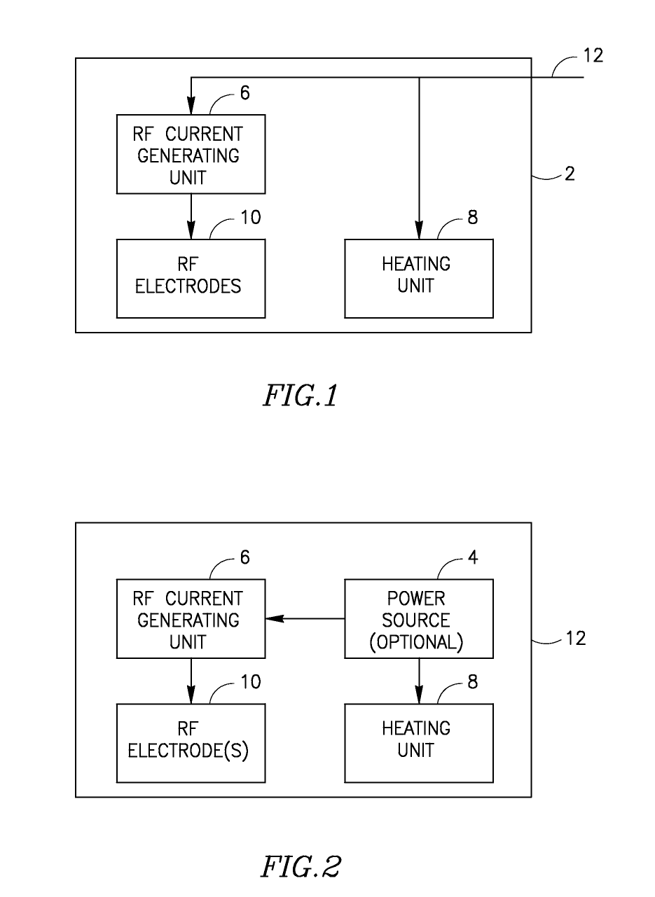

Reference is now made to FIGS. 1 and 2 which are schematic block diagrams illustrating devices for hair removal in accordance with an embodiment of the present invention. The device 2 (of FIG. 1) includes an RF current generating unit 6, RF electrodes 10 for applying RF energy to the skin, a heating unit 8 for heating and/or cutting hair. In accordance with one embodiment of the invention, the RF current generating unit 6 and the heating unit 8 may be connectable to an external power source (not shown in detail in FIG. 1) such as, for example, through a suitable electrically conducting line 12 connectable to the mains AC supply.

The device 12 of FIG. 2 includes (in addition to the RF current generating unit 6, the RF electrodes 10 and the heating unit 8) an internal power source 4 for providing power to the heating unit 8 and to the RF current generating unit 6.

The power source 4 of FIG. 2 is suitably coupled to the RF current generating unit 6 and to the heating unit 8 to supply power thereto as is known in the art. The power source 4 is preferably an electrical power source such as, but not limited to a DC (direct current) power supply or an AC (alternating current) power supply, a fuel cell, a primary or a rechargeable electrochemical cell or any other type of suitable electrical power source known in the art.

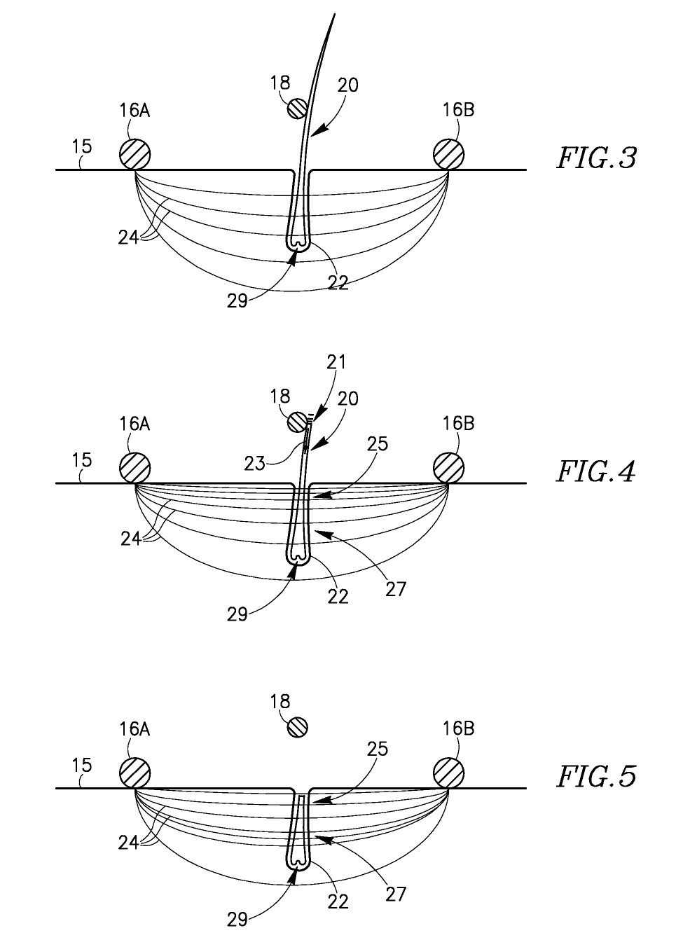

The RF electrodes 10 may be any suitable RF electrodes adapted to applying RF currents to the skin. The RF electrodes 10 may be implemented as two or more electrically conducting members adapted for contacting the skin and passing RF currents through the skin. Typically, the RF electrodes 10 may be arranged in a bipolar arrangement (see FIG. 3 below) as is known in the art (for example U.S. Pat. No. 6,889,090 to Kreindel discloses bipolar RF electrode arrangements). However, the RF electrodes 10 may also be configured in a unipolar arrangement (not shown) where one electrode (also known as the “active electrode”) is put in contact with the skin at the site treated for hair removal while the other electrode (also known as the “return electrode”) may be put at a place relatively distant from the treated site (such unipolar RF electrode arrangements are often used in surgical RF cauterizing devices) as is known in the art.

Reference is now made to FIGS. 3-5 which are schematic cross-sectional diagrams illustrating different successive stages of a skin region during treatment by a hair removal device of the present invention having a single heating element and a bipolar RF electrode arrangement, in accordance with an embodiment of the present invention. The RF electrodes 16A and 16B are configured as two electrically conducting metallic rods (shown in cross-section) which are in contact with the surface of the skin 15. The heating unit of the device is implemented as a thin electrically conducting metallic wire 18 (shown in cross-section). The wire 18 is shown touching the shaft of a hair 20 which is disposed in the hair follicle 22. When RF current is passed between the RF electrodes the current flows through the skin (as schematically represented by the lines 24) causing the skin to be heated to a temperature below the coagulation temperature of the living tissue (not shown in detail) of the follicle 20.

Turning to FIG. 4, when a suitable electrical current (DC current or AC current may be used) is passed through the wire 18, the wire 18 heats up due to its resistance. If the temperature of the wire 18 reaches or exceeds the hair ignition temperature (which occurs roughly at about 450° C.), the hair shaft 20 is heated by the wire and is ignited near or at the point of contact 21 between the hair shaft 20 and the hot wire 18. The ignited hair shaft 20 burns (the upper part of the hair shaft 20 is cut off and is therefore not shown in FIG. 4) and the burning of the hair shaft 20 proceeds towards the skin 15 in the general direction represented by the arrow 23. As the hair shaft 20 continues to burn and the remaining hair shaft stub shortens, the upper part of the skin in the vicinity of the lower part of the remaining stub of the hair shaft 20 and some of the tissues of the follicle 22 become further heated due to heat conduction along the remaining portion of the hair shaft 20. Thus the temperature of the skin region 25 increases. Because the impedance of the skin and tissues of the follicle 20 decreases as the tissue temperature increases (up to the tissue coagulation temperature), the hotter region 25 now has a lower impedance (to current in the RF range) and the RF current density in the hotter region 25 increases (which is schematically represented by the higher density of the of lines 24 within the region 25) which in turn causes a higher RF energy dissipation in the region 25 and a further increase in the local temperature of the region 25. This positive feedback effect causes the temperature of the region 25 to further increase until the temperature reaches the tissue coagulation point and the tissue in the region 25 begins to coagulate.

As the impedance (in the RF range) of coagulated tissue is now much higher than the impedance of non-coagulated tissue, the coagulation of the tissue in the region 25 results in increased impedance of the region 25 to RF current flow and reduces the RF energy dissipation into the region, effectively stopping the positive feedback effect within the region 25. However, heat flows from the coagulated region 25 deeper into the skin towards a deeper region 27 adjacent to the region 25.

Turning to FIG. 5, the RF current density in the coagulated region 25 is now lower (because of tissue coagulation), while the positive feedback effect has now moved or migrated to the region 27 further heating up the region 27 and reducing its impedance (as represented by the higher density of current lines 24 in the region 27). It is noted that the burning of the hair shaft 20 has stopped (probably due to lack of oxygen and heat dissipation in the skin tissues surrounding the shaft 20). Typically, (based on visual observations and photographic results) the hair shaft 20 may stop burning at a point approximately 0.25 millimeters below the surface of the skin 15, but this depth may vary between individuals and between different hair types or different regions of skin treated.

As the region 27 reaches the tissue coagulation temperature, its impedance begins increasing again and the region of high current density moves deeper in the general direction represented by the arrow 23 of FIG. 4.

As schematically illustrated in FIGS. 3-5, a “hot spot” or moving region of low impedance and higher temperature advances along of the skin tissues surrounding the hair shaft 20 in the general direction towards the hair papilla 29 situated at the base of the follicle 22. This region may also be defined as a moving coagulation zone. As this hot spot or moving coagulation zone advances, the tissues affected by it become fully or partially coagulated. This coagulation affects the ability of the cut hair to re-grow, resulting in a more efficient depilation effect and preventing or at least significantly reducing re-growth of some of the cut hairs.

It is noted that while the moving coagulation region phenomenon described herein occurs more intensively when the hair shaft 20 is actually ignited and burns, it may also occur in cases in which the temperature of the wire 18 is below the hair ignition temperature. In such cases while the hair shaft 20 is not cut, a moving tissue coagulation region or zone may still be formed and may propagate in a skin tissue region adjacent to the hair shaft 20 due to some heat conduction along the hair shaft 20.

The implementation of the hair removing devices of the present invention may vary depending upon the specific application. For example, the hair removal device may be implemented as a desktop or bedside system for use by a physician or cosmetician or another user. Such a system may include a base station for housing some of the necessary electrical circuitry for providing power and control functions, safety functions and/or other components of the system and a hand-held part (also referred to as a hand-held applicator throughout the present application) which may be applied to the treated skin area and which may include the RF electrodes and the heating element or heat source, and possibly some additional components (such as but not limited to, sensors).

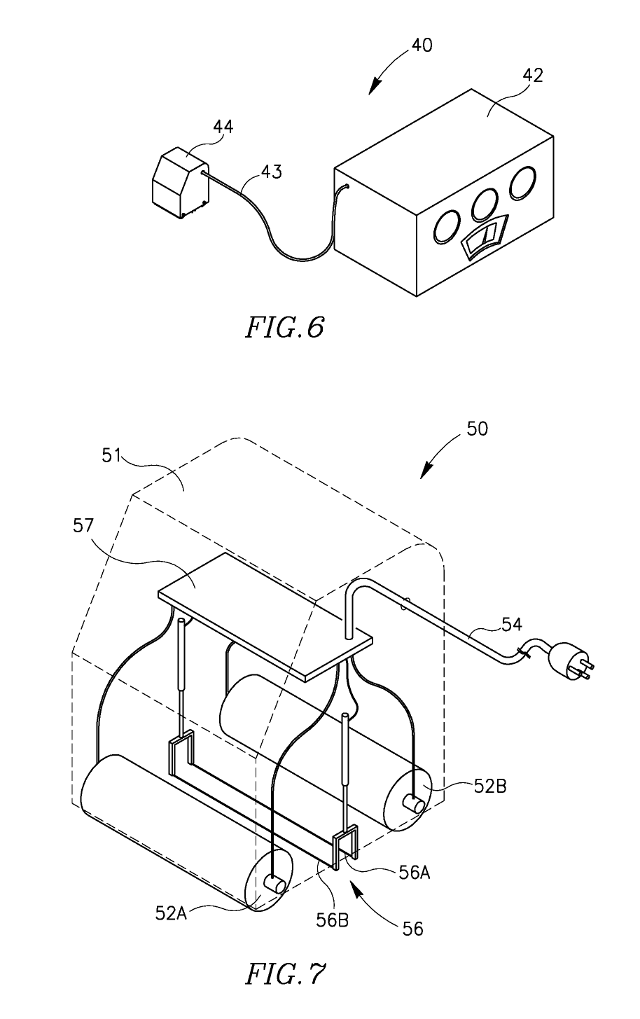

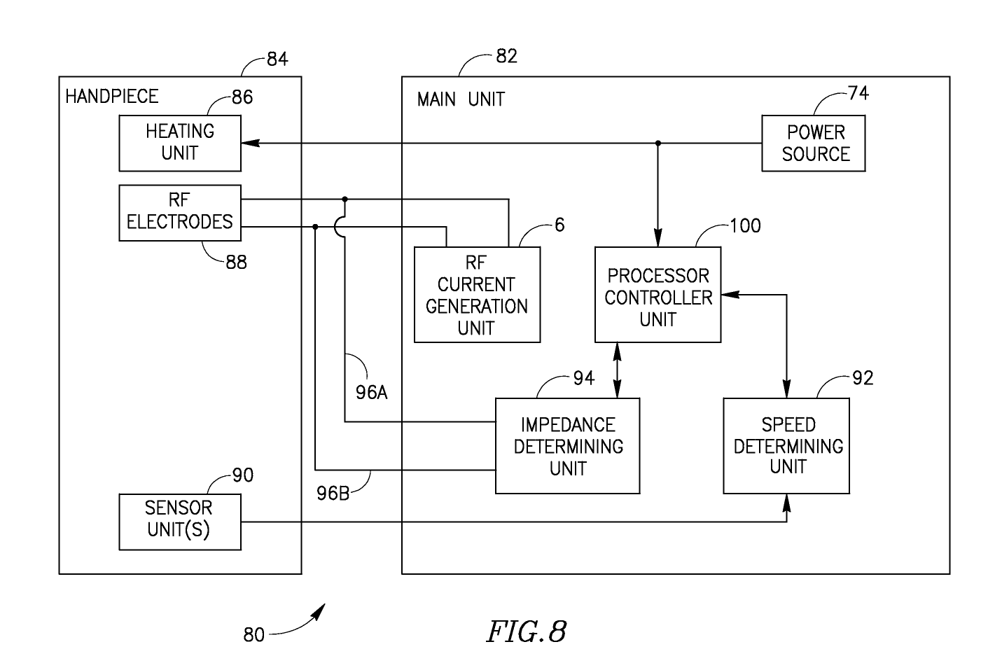

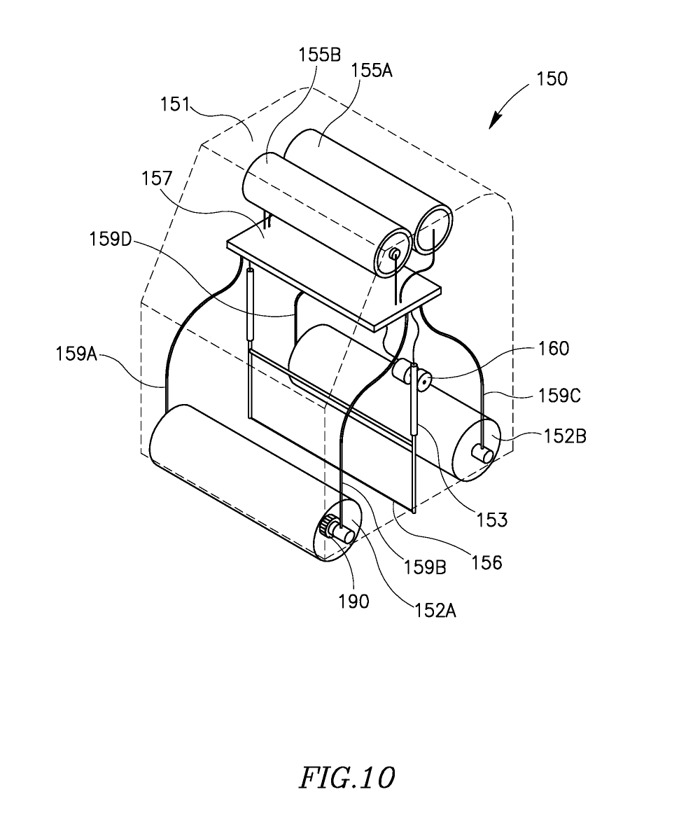

Reference is now made to FIG. 6 which is a schematic isometric view illustrating a hair removal system including a base station and a hand held part, in accordance with an embodiment of the present invention. The system 40 includes a base station 42 and a hand-held part 44 connected to the base station 42 by a suitable connecting cable 43. The system 40 also includes a hand-held part 44 which may include RF electrodes for applying RF energy to the skin (the RF electrodes of the hand-held part 44 are not shown in detail in FIG. 6 for the sake of clarity of illustration and may be similar in structure and operation to the RF electrodes 52A and 52B of the device 50 of FIG. 7 below, or to the RF electrodes 152A and 152B of the device 150 of FIG. 10 below, or to any type of RF electrodes described herein or known in the art). The hand held part 44 also includes a heating unit including one or more heating elements for heating and/or cutting hair and for initiating the coagulation cascade wave as described hereinabove in at least some of the cut and/or heated hairs. The heating unit is not shown in detail in FIG. 6 for the sake of clarity of illustration and may be similar in structure and operation to the heating unit 56 of the device 50 of FIG. 7 or to the movable heating unit 153 of the device 150 of FIG. 10, or to any other type of heating unit described herein or known in the art), The base station 42 includes a suitable RF current generating unit (not shown in FIG. 6, for the sake of clarity of illustration) for energizing the RF electrodes of the hand-held part 44. The base station 42 may also include a power source (not shown in detail in FIG. 6 for the sake of clarity of illustration) for energizing the heating elements of the heating unit and/or for providing power to control circuitry (not shown in detail for the sake of clarity of illustration) which may be included in the base station 42 or (alternatively or additionally) in the hand-held part 44 for controlling various treatment parameters and for operating various safety features (such as but not limited to sensor(s), a velocity determining unit, an impedance determining unit, a controller processor unit, and the like, as described in more detail in the system 80 of FIG. 8 hereinbelow). The power source may be an internal power source included in the base station 42 (not shown in detail in FIG. 6 for the sake of clarity of illustration, but similar to the power source 4 of FIG. 2 or the power source 74 of FIG. 8 or the batteries 155A and 155B of FIG. 10, or may also be implemented as any other suitable electrical power source known in the art and described herein) or may be the mains AC power supplied to the base station through a suitable mains power cable (not shown).

It is noted that in other embodiments in which the hand-held part 44 includes only active RF electrode(s), the system 40 may include a return RF electrode (not shown in FIG. 6) which may be suitably electrically connected to a distant part of the treated skin to serve as a return path for the RF currents. For example, such a return RF electrode (not shown) may be connected to the base station 42 by a suitable isolated electrically conducting wire or cable (not shown).

Reference is now made to FIG. 7 which is a schematic isometric view of a hand held hair removing device, in accordance with another embodiment of the present invention. The hand held device 50 includes a housing 51, a pair of RF electrodes 52A and 52B fixedly or movably attached to the housing 51 and arranged in a bipolar arrangement such that both of the RF electrodes may be placed in contact with the skin (not shown). The device 50 has a power cord 54 that may be suitably connected to a mains AC power socket to receive power for operating the device 50. Alternatively, in accordance with another embodiment of the device, the power cord 54 may be eliminated and the internal circuitry of the device may be powered by using a suitable internal power source (not shown) included within the housing 51. The internal power source (not shown) may be any suitable electrical power source, such as but not limited to a battery or any other suitable electrochemical cell (primary or rechargeable), or a fuel cell, or any other suitable compact electrical power source known in the art. The device 50 also includes a heating unit 56. The heating unit 56 includes heating elements 56A and 56B, such as but not limited to a metallic wires (such as but not limited to wires made of a Nickel Chrome alloy, or of any other suitable metallic or non-metallic electrically conducting material) or other suitable forms of electrically conducting element(s) configured for heating and/or cutting hairs of the skin. The heating elements 56A and 56B may be rigidly attached to the housing 51 but may also be movably attached thereto, such that the distance between the skin and the heating elements 56A and 56B may be varied. Additionally, the heating elements 56A and 56B or parts thereof may be detachably attached to the housing 51 such that it may be periodically replaced. The device 50 further includes a circuit board (or circuit boards) 57 which includes the necessary RF current generating circuitry (not shown in detail) and suitable circuitry for providing electrical energy to the heating elements 56A and 56B (either DC current or AC current). It is noted that while the specific implementation of the device 50 of FIG. 7 includes two heating elements 56A and 56B therein, any suitable number of wires (including a single wire or heating element, and more than two wires or heating elements) or any other type of heating elements may be used in implementing the device of the present invention (including a single heating element implementation).

It will be appreciated by those skilled in the art that the heating unit of the present invention is not limited to the implementation including a heated wire or a plurality of heated wires. Rather, any type of heating element (or heating elements) known in the art may be used for cutting and/or igniting and or heating the hair. For example, the heating unit of the devices of the present invention may include any type and suitable number of heating elements, including but not limited to ceramic heating element(s) metal coated ceramic element(s) metallic or other electrically resistive ribbon-like heating element(s) or any other type of suitable heating element(s) known in the art.

It is noted that the devices of the present invention may also include various types of components and/or circuitry for implementing various safety features to avoid applying excessive amounts of RF energy to the skin and to control the application of heat by the heating element(s) of the devices.

Reference is now made to FIG. 8 which is a schematic diagram illustrating a system for hair removal including various safety features, in accordance with an embodiments of the present invention.

The system 80 includes a main unit 82 and a hand-piece 84. The hand-piece 84 includes a heating unit 86 which may include one or more heating elements (not shown in detail in FIG. 8) as described hereinabove. The hand-piece 84 also includes RF electrodes 88. The RF electrodes are preferably arranged in a bipolar configuration (see FIG. 7 hereinabove) but a unipolar arrangement may also be implemented.

A common problem encountered when RF electrodes are used to heat the skin by delivering RF energy to the skin is that the electrical coupling of the electrodes to the skin may not always be optimal. For example, if the RF electrodes 88 are not well coupled electrically to the underlying skin sparking may occur between the electrodes and the skin which may be undesirable due to increased possibility of burning of the skin. Therefore, in accordance with an embodiment of the present invention, a safety mechanism may (optionally) be included in the systems or devices of the present invention to ensure proper electrical coupling of the RF electrodes to the skin during the delivery of RF currents.

The Main unit 82 also includes an RF current generating unit 6 (see also FIG. 2), suitably electrically coupled to the RF electrodes 88 of the handpiece 84. The RF current generating unit may be any type of suitable RF current generating unit known in the art. The RF current generating unit 6 may be a variable power unit which may be controlled by a suitable user interface (The user interface is not shown in FIG. 8 for the sake of clarity of illustration, but may be implemented as one or more of the dials or controlling elements illustrated in the main unit 42 of FIG. 6). Typically, but not obligatorily, the energy applied to the skin by the RF current generating unit 6 may be controlled by controlling the voltage applied to the electrodes 88 or by controlling the duty cycle of the pulsed RF currents generated by the RF current generating unit 6. It may be possible to control or vary the RF pulse duration or the pulse repetition rate or both the RF pulse duration and the Pulse repetition rate to control the amount of RF energy applied to the skin through the RF electrodes 88. However, the person skilled in the art will appreciate that any suitable type of RF current generating unit may be used in the present invention, and that other suitable methods for controlling the application of RF energy to the skin may be used.

The main unit 82 may also include a power source 74. The power source 74 supplies power to the heating unit 86 and to all the electrical components included in the main unit 82 and/or to any other electrical component requiring power in the handpiece 84, such as, for example to the sensor unit(s) 90. The power source 74 may be any suitable type of power source known in the art (AC and/or DC power sources may be used) and may be disposed within the main unit as shown, or alternatively be located outside the main unit 82. It is noted that for the sake of clarity of illustration, the connections of the power source 74 to the various components of the main unit 82 and the handpiece 84 are not all shown in detail.

The main unit 82 also includes a processor/controller unit 100. The processor/controller unit 100 may be any suitable processor or controller or microprocessor or digital signal processor unit known in the art. The processor/controller unit 100 may be (optionally) suitably connected to the RF current generating unit 6 for controlling the operation of the RF current generating unit 6, (the connection is not shown in FIG. 8 as it is optional). If the processor/controller unit 100 is connected to the RF current generating unit 6, the processor controller unit 100 may also be connected to a suitable user interface unit (not shown in FIG. 8) such as, for example, the dials included in the main unit 42 of FIG. 6. The user interface unit may be used by a user of the system 80 to provide control signals either directly to the RF current generating unit 6, or alternatively to the processor/controller unit 100. Such control signals may control the application of RF energy to the skin as described.

The system 80 may also (optionally) include an impedance determining unit 94. The impedance determining unit 94 may be suitably coupled to the RF electrodes 88 by electrically conducting wires 96A and 96B. The impedance determining unit 94 may be suitably coupled to a processor/controller unit 100 and may provide the processor/controller unit 100 with data or signals representing the inter-electrode impedance value measured between the RF electrodes. The impedance determining unit 94 may measure the impedance to current flow (preferably, but not obligatorily, in the RF frequency range of 300 KHz-100 MHz) between the electrodes by using any suitable methods for impedance determination known in the art. For example, the impedance measurement methods disclosed in U.S. Pat. No. 6,889,090 to Kreindel, incorporated herein by reference in its entirety, may be used for impedance determination. However, any other suitable impedance measurement known in the art may be used in implementing the present invention.

If the measured impedance is less than or equal to a certain preset or predetermined value, the impedance determining unit 94 or the processor/controller unit 100 may enable the application of RF currents to the RF electrodes 88. If the impedance value determined by the impedance determining unit 94 exceeds the preset or predetermined value, the impedance determining unit 94 or the processor/controller unit 100 may disable the application of RF currents to the RF electrodes 88. This safety mechanism may thus prevent or at least reduce any sparking that may be caused by an increase in the impedance due to unsuitable positioning or placement of the hand-piece 84 on the skin or insufficient pressure applied to the hand-piece 84 or any other reason causing an increase in the inter-electrode impedance. Typically, suitable impedance values for safe delivery of RF currents in the RF range of (300 KHz-100 MHz) is about several hundreds ohms. Thus, in a non-limiting example, for a pulsed RF application with an RF frequency of 1 MHz, an RF pulse repetition rate (PRR) of 100 Hz and an RF pulse duration of 2 milliseconds applied to typical skin, a threshold value of 500 ohms for the inter RF electrode impedance may be used as the threshold value for increasing safety of use. If the selected impedance threshold value is 500 ohms, during use of the handpiece 84, if the measured impedance between the electrodes 88 exceeds 500 ohms, the supply of RF currents through the electrodes 88 is disabled. For measured impedance values less than 500 ohm the RF currents are enabled.

It is noted that in accordance with an embodiment of the present invention the impedance threshold value may be a preset value (which may be set at the factory). However, preferably, in systems such as (but not limited to) the system 80 which have a controller unit or a processor unit such as the processor/controller unit 100, the user of the system may set the impedance threshold value by using a suitable user interface (not shown), such as, for example a suitable dial (not shown in FIG. 8), a keypad (not shown in FIG. 8) or any other suitable user interface device suitable for providing user input or control signals to the processor/controller unit 100. For example, such an (optional) user interface may be included in the main unit 82 or in the handpiece 84, or in both (see, for example, the dials of the main unit 42 of FIG. 6).

The user of the system 80 may thus be able to use the user interface to set the impedance threshold value to any desired value within an allowed impedance range. Typically, such an allowed impedance threshold value range may be 200-1000 Ohm. However, other different impedance threshold ranges may also be used depending on the application. Such user control of the impedance threshold value may be useful because the properties of the skin may vary from person to person, or for the same person at different times (due to possible changes in the skin's condition such as, inter alia, different states of skin hydration and electrical conductivity, the person's physiological condition and the like) and even in different body parts of the same person. Thus, the system 80 may allow the user to adjust the impedance threshold value in order to adapt the system for use with different patients or with different skin conditions or for different properties of the skin in different treated body parts of the same patient.

It is, however noted, that the threshold value is not limited to the exemplary threshold value disclosed hereinabove and that other different threshold values may also be used in different embodiments of the present invention.

Another (optional) safety mechanism may be used to avoid excessive application of heat by the heating unit 86 in cases in which the speed of movement (velocity of the handpiece relative to the treated skin is too low. Thus, the hand-piece 84 may also (optionally) include one or more sensor units 90.

The sensor(s) 90 may be any sensor suitable for detecting the speed of motion (velocity) of the hand-piece 84 along the skin. It is noted that methods and sensors for determining the velocity or speed of movement of a handpiece or a device relative to the skin are well known in the art, are not the subject matter of the present invention and are therefore not disclosed in detail hereinafter. For example, methods and devices for such velocity determination may be implemented using a mechanical gyro (see, for example U.S. Pat. No. 5,296,794), an optical gyro (see, for example U.S. Pat. No. 4,514,088), an optical mouse (see, for example U.S. Pat. Nos. 4,631,400 and 4,920,260), other mechanical systems such as encoders (see, for example U.S. Pat. Nos. 5,235,514, and 5,208,521), all of the above cited patents are incorporated herein by reference in their entirety. However, it is noted that other suitable types of sensors and velocity determining methods known in the art may be used for implementing the velocity determination of the devices of the present invention and may be easily adapted for use in the present invention by those skilled in the art.

Typically, when the sensor(s) 90 include velocity sensors, the velocity sensors may be suitably connected to an (optional) speed determining unit 92 included in the main unit 82 (of FIG. 8). The speed determining unit 92 may receive signals from one or more of the sensor(s) 90 and may process the signals to determine the speed of movement (velocity) of the hand-piece 84 relative to the skin (not shown in FIG. 8). If the measured speed of movement of the hand-piece 84 relative to the skin is lower than a preset or predetermined speed threshold value, the supply of current to the heating unit 86 may be interrupted to avoid excessive heating of the skin by the heating unit 86 in a slow moving or stationary hand-piece 84. The speed determining unit 92 may be coupled to the processor controller unit 100 (as shown in FIG. 8) or may also be coupled to the power source 74 to control the application of electrical current from the power source 4 to the heating unit 86.

Typically, RF current parameters which may be used in the present invention include, but are not limited to radio frequencies in the range of 200 KHz-100 MHz, (and preferably in the range of 300 Khz-10 MHz) and currents in the range of 10 mA-1.0 A. For pulsed RF, the pulse repetition rate may be in the range of 100 Hz-25 KHz and RF pulse duration may be in the range of 0.01-2 milliseconds. It will be appreciated by those skilled in the art, that other, different values or different ranges of the RF pulse repetition rate parameter and the RF pulse duration may be used, depending, inter alia, on the application type, the RF source type, the presence or absence of active skin cooling, and on other practical considerations.

It is further noted that while preferably, the RF energy application mode used in the methods, devices and systems disclosed herein comprises delivering to the skin pulsed RF energy or Quasi-continuous RF energy (quasi-CW RF energy) by applying to the RF electrodes pulsed RF voltage or Quasi-continuous RF voltage, respectively, it is also possible to use a continuous wave RF (CW-RF) energy delivery mode by applying to the RF electrodes continuous wave RF voltage.

Furthermore the parameters of RF energy delivery to the skin and RF voltages applied to the RF electrodes may be any of the parameters disclosed in US published Patent Application, No. 2006/0173518 to Kriendel entitled “Device and Method for treating skin”, incorporated herein by reference in its entirety.

It is noted that the power rating of the RF current generating units (such as, but not limited to the RF current generating unit 6 of FIGS. 1, 2 and 8) usable for implementing the methods, devices and systems for hair removal described herein may typically be in the range of 1-20 Watt (nominal RF power rating) and preferably in the range of 2-10 Watt (nominal RF power rating). However, it will be appreciated by those skilled in the art that RF generating units having other different RF power rating values (which may be smaller than 1 Watt or much larger than 20 watt) may also be usable in the devices and systems, depending, inter alia, on the type of device used, the type of the RF currents being used (Pulsed RF, CW-RF or quasi-CW RF), the (optional) use of cooling methods (such as, but not limited to the cooled RF electrodes described hereinbelow), the region of skin to be treated, the (optional) use of anesthetic compositions on the treated skin (which may enable using higher levels of RF energy delivery to the treated skin while keeping patient discomfort at reasonable levels), and other considerations. Furthermore, in devices using unipolar RF electrode arrangements, the RF generating units may have a power rating much larger than the typical power ratings disclosed above. Thus the typical range of 1-20 watt is given by way of example only and is not intended to imply an upper or lower limitation to the RF power usable in implementing the methods, devices and systems disclosed herein.

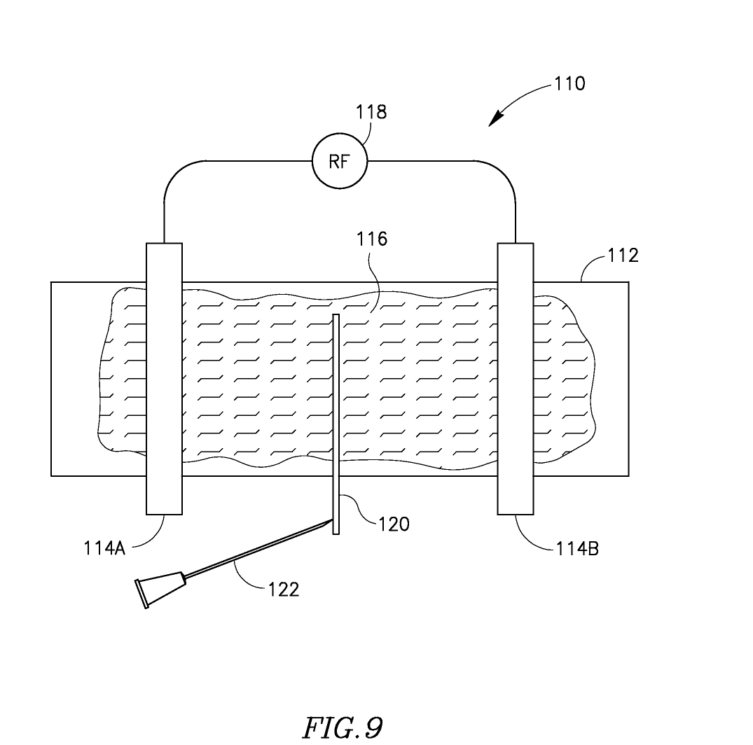

Reference is now made to FIG. 9 which is a schematic top view of an experimental device for demonstrating and testing the heat induced RF coagulation cascade effect used in the present invention. The experimental device 110 included a microscope glass slide 112. A pair of RF electrodes 114A and 114B was placed on the glass-slide 112. The electrodes 114A and 114B were cylindrical electrodes made of bronze metal having a cross-sectional diameter of four millimeters. The distance between the electrodes 114A and 114B was 30 millimeters An egg-white sample 116 (a few drops of fresh uncooked egg-white) was placed on the slide 112 such that the electrodes 114A and 114B were fully immersed in the egg-white sample 116 to ensure good contact between the electrodes 114A and 114B and the egg-white sample 116. An RF generator 118 was connected to the electrodes 114A and 114B.

The electrodes 114A and 114B were connected to an RF generator 118 (Model SURTRON 80, commercially available from LED SpA, ITALY). An RF power of increasing magnitude in the range of 6-10 W was applied between the electrodes 114A and 114B while visually watching the egg-white sample 116 until the egg-white sample 116 became opaque white due to coagulation caused by RF induced heating. In the experiments described herein, the egg-white sample 116 was used to simulate the skin cells or skin tissue(s) surrounding a hair shaft of a human hair disposed in the skin. The experiment was repeated several times to determine suitable RF current conditions for which there was no observable coagulation of the egg-white sample 116 for a prolonged (about 10-15 minutes time period) application of the RF current. It was found that RF power of values of about 10 W could be routinely applied to the egg-white sample 116 without any observed coagulation, indicating that the rate of dissipation of RF current generated heat by the glass slide 112 and RF electrodes 114A and 114B (and possibly by air surrounding the experimental setup 110) was enough to keep the temperature of the egg-white sample 116 sufficiently below the coagulation temperature of egg-white for the tested time periods. Thus, at these specific RF power conditions, the equilibrium (steady state) temperature of the egg-white sample 116 was below the coagulation temperature of egg-white and no coagulation occurred during the RF power application time period.

After determining the conditions under which such RF currents did not cause immediate coagulation of the egg-white sample 116, a series of additional experiments was performed. In each of the new experiments, a human hair 120 having a total length of about 50-70 millimeters was immersed in the fresh sample of egg-white 116 (it is noted that the sample of egg-white 116 was replaced with a fresh new sample after each experiment was completed, and a new different human hair was used for each experiment) about mid-way between the RF electrodes 114A and 114B. The RF power level was set to 10 W and current was applied between the Electrodes 114A and 114B for approximately three minutes while the egg-white sample was continuously visually observed. No coagulation was observed in this first observation time period. At the end of the three minutes observation period a hot needle 122 at a temperature of about 500-600° C. was put in contact with the end of the human hair protruding over the edge of the glass slide 112 (as illustrated in FIG. 9) without changing the parameters of the RF current applied to the egg-white sample 116. Very fast coagulation of the egg-white was observed along the hair 120 immediately after the hot needle 122 was put in contact with the end of the human hair 120. The egg white adjacent the hair 120 turned from transparent to opaque white immediately after the contact of the hot needle 122 with the hair 120 indicating very rapid coagulation of the egg-white near the hair 120. The coagulation was filmed with a video camera and individual frames of the resulting video data were displayed and visually examined. From the observed frames, it became apparent that the “cascade” or positive feedback effect in which the temperature of the egg-white sample surrounding the hair shaft quickly increased due to lowering of the resistance to RF currents in the portion of egg-white adjacent to the heated hair 122 causing a positive feedback with increasing deposition of RF energy into the egg-white surrounding the hair shaft was very fast and the transition between a transparent egg-white through which the dark pigmented hair could be clearly observed to an opaque white coagulated egg-white completely covering the hair, occurred within a single video frame time.

The results of the above experiments demonstrate the feasibility of achieving hair removal and reduced hair re-growth through the combining of two synergistic actions: a localized heating and/or cutting of the hair shaft(s) using a heat source and the application of RF energy to the skin (in the form of RF currents) which provides additional energy for coagulating the tissues adjacent to the heated part of the hair shaft(s).

The localized heating preferably involves the heating and/or cutting of hair by applying to the hair shaft a heat source (such as, but not limited to, a hot wire). The heat source preferably has a temperature sufficient to ignite the hair shaft effectively cutting the shaft but it is also possible to use a heat source temperature lower than the hair ignition temperature such that the hair shaft is heated without being cut.

As discovered by the inventors of the present invention, the combination of the localized heating and/or cutting of the hair(s) with the application of RF currents to the skin at suitable RF current intensity levels results in the formation of a spreading or moving coagulation wave (cascade) in the skin tissues close to the hair which effectively reduces hair re-growth. The RF currents are applied to the skin at a current intensity which by itself (taken alone, without the localized heating of the hair shafts by the heating unit) would not be sufficient to heat the skin tissue to a temperature required for coagulation of the tissues surrounding the hair shafts (such as the hair follicle tissues). However, the additional heating of hair shafts ignited (or heated without being ignited) by contact with the heating element (such as, but not limited to, the wire 18 of FIGS. 3-6 or the heated needle 122 of FIG. 9 or any other heating element used in the various described embodiments of the present invention) initiates the tissue coagulation cascade caused by positive feedback as described above.

Without being bound by a specific theory, it is possible that the coagulation of the skin tissue adjacent the hair shaft (such as, for example, the hair shaft 18 of FIG. 3) may contribute to the long term depilation effect, possibly by disrupting tissues and cells associated with hair growth and/or with the natural hair growth controlling cycle.

Reference is now made to FIG. 10 which is a schematic isometric view illustrating a battery operated hand-held hair removing device having a movable heating unit, in accordance with another embodiment of the present invention. The device 150 includes a pair of RF electrodes 152A and 152B rotatably attached to a housing 151. The housing 151 includes a pair of batteries 155A and 155B which are suitably electrically connected to an electronic circuit board 157. The electronic circuit board 157 may include suitable current and voltage converting circuitry (not shown in detail) for generating RF currents.

The circuit board 157 may also include a suitable controller or processor (not shown). The RF electrodes 152A and 152B are suitably electrically connected to RF generating circuitry on the board 157 through suitable conducting wires 159A, 159B, 159C and 159D. A heating element 156 (a nickel-chrome wire may be used, but other types of suitable heating elements may also be used) is connected to the circuit board 157 which provides DC or AC currents for heating the heating element 156. The heating element 156 is attached to a movable heating unit 153 which is mechanically coupled to an electrical motor 160.

The motor 160 is controlled by the circuitry of the board 157 to controllably move the heating unit 153 and the heating element 156 attached thereto. The circuit board 157 may (optionally) include the impedance determining unit 94 and the speed determining unit 92 of FIG. 8. The device 150 may also include a sensor 190 for determining the speed of movement of the device 150 relative to the skin. The sensor 190 may be any suitable sensor adapted to determine the rate of rotation of the RF electrode 152A and to provide a signal to a processor unit (not shown) included in the board 157.

When the circuitry board 157 (or a processor or controller included therein) determines that the device 150 is stationary relative to the skin, based on signals or data received from the sensor 190, the motor 160 may be activated to lift the heating unit 153 and the heating element 156 off the skin to prevent burning of the skin by the heating element 156 (the circuit board 157 may also, optionally, switch off the current flow though the heating element 156). When the circuit board 157 determines that the device moves at a suitable speed relative to the skin, the motor may be activated to lower the heating unit 153 towards the skin (and may, optionally, also enable the flow of current through the heating element 156). It is noted that the heating unit 153 and/or the heating element 156 may be fixed units or alternatively may be detachable or removable or replaceable or disposable units, for ease of repairing and/or maintenance of such units.

Thus, when the heat source (or heating unit) of the present invention is used for cutting hair in conjunction with the application of RF currents to the skin as described herein, the hair is not only cut (shaved) but the resulting coagulation of tissues adjacent to the shafts of cut (ignited) hairs (and possibly of hairs heated by the heating unit without being ignited) may significantly contribute to the long term effectiveness of hair removal by reducing or preventing re-growth of the affected hairs.

It is noted than accordance with other embodiments of the hair removal device more than one sensor may be used for determining the velocity of the device relative to the skin. For example, in an additional embodiment, the device 150 may include an additional sensor (not shown in FIG. 10) similar to the sensor 190 which may be connected to the RF electrode 152B and may provide an independent output signal to the circuit board 157. Such embodiments with multiple sensors may be useful due to increased component redundancy and may be able to be safely operated even if one of the velocity sensors is temporarily or permanently disabled or provides an erroneous result. Alternatively, to increase safety, the heating unit of the device may be lowered towards the skin (and/or heated by applying electrical current thereto) only if both sensors provide signals indicative of a device velocity value which exceeds the velocity threshold.

Additionally, in contrast to many hair removal methods based on selective light absorption by the hair pigments which are not very efficient in patients having dark skin or pigmented skin regions, and/or grey, white or other light colored hairs, the devices, systems and methods of hair removal disclosed herein are highly efficient for hair removal from dark and pigmented skin and are efficient in removing white or grey or other light colored hairs.

It will be appreciated by those skilled in the art that many variations and permutations may be used in implementing the devices of the present invention. For example, while the heating element(s) used in the heating unit for cutting or heating hair are preferably electrically resistive metallic wires (such as, but not limited to suitable Nickel-Chromium wires), other heating elements may also be used having different composition, geometry and configuration. For example, it may be possible to use ceramic heating element(s) (such as, but not limited to, silicon carbide heating elements, or any other suitable ceramic heating elements known in the art and having a suitable electrical resistance value), ceramic elements with a suitable electrically resistive metallic coating, ribbon like heating elements (such as but not limited to metallic ribbons or thin flat ceramic resistive elements, or thin flat ceramic elements having a metallic resistive coating, or the like) or any other suitable type of heating element known in the art. It is noted that any suitable combination of such different heating elements may be used for implementing the heating unit(s) of the devices and systems described herein. For example, the heating unit 8 may include a single Nickel-Chrome wire, a single Nickel-Chrome ribbon, a single silicon carbide (SiC) element (having a rod like or flat shape), a single heat resistant ceramic element having a metalized surface, or a combination of any suitable number of such metallic and/or ceramic and/or metalized ceramic elements.

Additionally, the number and/or configuration and/or spatial arrangement of the heating element(s) included in the heating unit of the present invention may vary. For example, while a single heating element (such as but not limited to a heated metallic wire) may be used in the heating unit, any other suitable number of such heating elements may be used within a heating unit of the present invention (For example, it may be possible to use any of the heating element configurations or combinations disclosed in U.S. Pat. No. 6,825,445 to Shalev et al., incorporated herein by reference in its entirety, in implementing the heating unit(s) of the present invention).

Furthermore, the number, composition, dimensions, configuration and construction of the RF electrodes used in the devices of the present invention may be altered and varied according to the desired implementation and the application. For example, the RF electrodes may be static or rigidly attached to the housing of the hair removal device (not shown) or may be rotatably attached to the device such as the electrodes 52A and 52B of the device 50 of FIG. 7. The RF electrodes may be metallic electrodes but may also be made from or may include non-metallic electrically conducting parts or materials (such as, for example, electrically conducting polymers, graphite, carbon or the like).

It is noted that while the exemplary (non-limiting) devices 50 and 150 include two RF electrodes (arranged in a bipolar electrode configuration), this is not obligatory and any suitable type of electrode arrangement and number of electrodes known in the art may be used as may be easily understood and implemented by those skilled in the art. Thus, in accordance with another embodiment of the hair removal device, the device may include a plurality of (more than two) RF electrodes arranged in any suitable electrode configuration. For example, the device 50 may be modified by replacing the two RF electrodes 52A and 52B with four or six or any other suitable desirable number of) RF electrodes (not shown in detail). In embodiments of the device in which more than two electrodes are being used, the RF electrodes may be arranged in any suitable type of arrangement. For example, four electrodes may be configured as two bipolar electrode pairs, or as three active RF electrodes and a single return electrode, or any other suitable electrode configuration known in the art.

Further yet, the RF electrodes may be actively cooled electrodes (not shown). The cooling of the RF electrodes may be implemented by any suitable cooling mechanism known in the art. For example, one or more Peltier elements (not shown) may be used to cool the RF electrodes of the present invention, or the electrodes may be hollow electrodes and the electrode cooling may be achieved by passing or pumping a suitable coolant through such hollow electrodes. Methods for cooling the skin while performing hair removal treatments are well known in the art, are not the subject matter of the present application and are therefore not discussed in detail hereinafter. For example, any of the skin cooling methods described in U.S. Pat. No. 6,889,090 to Kreindel may be adapted for use in the systems and devices of the present invention. However, it is noted that many other suitable skin cooling methods and devices which are known in the art may be used in the systems and devices of the present invention.

The RF electrodes may be configured for improving the electrical contact between the RF electrodes and the skin. For example, the RF electrodes may be configured as hollow electrodes made from a porous material. Alternatively, at least one portion of the RF electrode may be a porous part (with or without the electrode being a hollow one). A reservoir (not shown) containing an electrically conducting solution (saline or the like) may be included in the device and suitably connected to the hollow part or porous part of the electrode(s). The electrically conducting solution may flow through the pores of the porous material of the RF electrodes (or the porous part of the electrode) to wet the surface of the RF electrodes to improve the electrical contact with the skin.

It is also noted that in accordance with an embodiment of the present invention the heating unit of the devices of the present invention need not be fixedly attached to the device. Rather, a replaceable and/or disposable or detachable heating unit may be used. In such an embodiment, it may be possible to detach the detachable heating unit from the device and replace it with a new heating unit (this may be useful if one or more of the heating elements of the heating unit is broken or becomes dysfunctional, less efficient, or burned.

It is also noted that in embodiments in which the hair removal device is a self-contained hand-held device, some or all the components included in any of the bedside or desktop systems described hereinabove (such as but not limited to the system 80 of FIG. 8) may be included within such a self-contained hand-held device. For example, in accordance with an embodiment of the device, the circuit board 157 of the device 150 (of FIG. 10) may also include the processor/controller unit 100, the RF current generating unit 6, the speed determining unit 92 and the impedance determining unit 94 of FIG. 8. In such a case, for example, the sensor 190 of the device 150 may be suitably connected to speed determining unit 92 or alternatively to the controller processor unit 100 for determining the velocity of the device 150 relative to the skin from the signals output by the sensor 190, and the motor 160 may be suitably connected to the controller processor unit 100 and may be controlled by the controller/processor 100 in accordance with the determined value of the velocity of the device 150, as described hereinabove.

Furthermore, it is noted that the various electronic and/or electrical components of all the systems and/or devices described herein may include any combination of analog components and/or digital components and/or hybrid analog/digital components. Further yet, the devices and systems described herein may be implemented by using any suitable combination of discrete electrical components and/or integrated circuits (including any type of IC or VLSI components known in the art.

It is noted that the various values of the RF current parameters disclosed hereinabove are given by way of example only and are not intended to limit the scope of the invention, while the values of the disclosed RF current parameters were found to be practical for working the invention, other different parameter values may also be used.

What is claimed is:

1. A device for hair removal, the device comprising:
a housing;
at least one RF electrode attached to said housing for applying RF electrical currents to a skin for heating at least part of said skin; and
at least one heating unit configured for applying heat to at least a portion of at least one hair of said skin.

2. The device according to claim 1 wherein said at least one RF electrode attached to said housing comprises an active electrode forming part of a unipolar RF electrode arrangement and wherein said unipolar RF electrode arrangement further includes a return RF electrode attachable to a distant part of said skin.

3. The device according to claim 1 wherein said at least one RF electrode comprises two RF electrodes arranged in a bipolar electrode configuration.

4. The device according to claim 1 wherein said at least one RF electrode comprises a plurality of RF electrodes attached to said housing.

5. The device according to claim 1 wherein said at least one RF electrode is electrically connectable to an RF current generating unit.

6. The device according to claim 5 wherein said RF current generating unit is selected from an RF current generating unit disposed within said housing and an RF current generating unit disposed outside of said housing.

7. The device according to claim 5 wherein said RF current generating unit has a nominal RF power rating in the range of 1-20 watt.

8. The device according to claim 5 wherein said RF current generating unit is configured for providing RF energy to said skin in an energy delivery mode selected from pulsed RF, continuous wave RF and quasi-continuous wave RF.

9. The device according to claim 5 wherein said RF current generating unit is configured for providing RF voltages having an RF frequency range of 200 KHz-100 MHz.

10. The device according to claim 6 wherein said RF current generating unit is an RF current generating unit disposed within said housing and wherein the device also includes an internal electrical power source for energizing said at least one heating unit and said RF current generating unit.

11. The device according to claim 10 wherein said internal electrical power source is selected from a direct current power supply, an alternating current power supply, a fuel cell, a battery, a primary electrochemical cell and a rechargeable electrochemical cell.

12. The device according to claim 6 wherein said RF current generating unit is an RF current generating unit disposed outside said housing and wherein said at least one heating unit and said RF current generating unit are energized by an electrical power source disposed outside said device.

13. The device according to claim 1 wherein said at least one heating unit is selected from a fixed heating unit, a movable heating unit, a removable heating unit, a detachable heating unit, a replaceable heating unit, a disposable heating unit and combinations thereof.

14. The device according to claim 1 wherein said at least one heating unit comprises one or more heating elements.

15. The device according to claim 14 wherein said one or more heating elements are selected from one or more electrically resistive wires, one or more metallic wires, one or more metallic ribbons, one or more ceramic heating elements, one or more metalized ceramic elements and combinations thereof.

16. The device according to claim 1 wherein said device is configured as a hand held device connectable to a base station.

17. The device according to claim 16 wherein said base station comprises an RF current generating unit for energizing said at least one RF electrode.

18. The device according to claim 16 wherein said base station comprises an electrical power source for energizing said heating unit.

19. The device according to claim 16 wherein said base station comprises a controller unit for controlling the application of RF energy to said at least one RF electrode.

20. The device according to claim 16 wherein said base station comprises a controller unit for controlling the heating of said heating unit.

21. The device according to claim 16 wherein said hand held device comprises one or more sensor units and wherein said base station comprises circuitry for receiving signals from said one or more sensor units and for processing said signals.

22. The device according to claim 21 wherein said at least one RF electrode comprises at least one active RF electrode, said base station further includes at least one return RF electrode, and said one or more sensor units comprise sensors for determining the impedance between said at least one active RF electrode and said at least one return RF electrode.

23. The device according to claim 21 wherein said one or more sensor units comprise sensors for providing signals representative of the velocity of said hand-held device relative to the skin.

24. The device according to claim 1 wherein said at least one RF electrode comprises at least two RF electrodes arranged in a bipolar configuration, said device also comprises an RF current generating unit controllably connectable to said at least two RF electrodes, said device also includes an impedance determining unit for determining the electrical impedance between said at least two RF electrodes, said device further includes a controller unit coupled top said RF current generating unit and to said impedance determining unit, said controller unit is configured for terminating the application of RF currents to said at least two RF electrodes when the impedance value between said at least two RF electrodes exceeds an impedance threshold value and for initiating the application of RF currents to said at least two RF electrodes when the impedance value between said at least two RF electrodes is equal to or smaller than said impedance threshold value.

25. The device according to claim 1 wherein said at least one RF electrode comprises at least one actively cooled RF electrode.

26. The device according to claim 25 wherein said at least one actively cooled RF electrode is selected from a hollow RF electrode having at least one passage therewithin for coolant flow within said RF electrode and an RF electrode comprising a Peltier element.

27. The device according to claim 1 wherein said at least one RF electrode comprises an RF electrode having at least one porous portion capable of containing an electrically conducting solution therewithin.

28. A system for hair removal, the system comprising:
RF electrodes for applying RF electrical currents to the skin;
an RF current generating unit controllably connectable to said RF electrodes; and
at least one heating unit for applying heat to at least a portion of at least one hair of said skin.

29. The system according to claim 28 also comprising a controller unit for controlling the application of said RF electrical currents to said skin.

30. The system according to claim 29 wherein said controller unit also controls the operation of said heating unit.

31. The system according to claim 28 also comprising a controller unit for controlling the operation of said heating unit.

32. The system according to claim 29 also comprising an impedance determining unit for determining the value of the electrical impedance between said RF electrodes, said impedance determining unit is connected to said controller and to said RF electrodes, said controller is configured for terminating the application of RF currents to the skin when the value of the impedance between said RF electrodes exceeds a threshold impedance value.

33. The system according to claim 29 also comprising a speed determining unit for determining the velocity of movement of said RF electrodes along the skin.

34. The system according to claim 33 wherein said speed determining unit is connected to at least one sensor.

35. The system according to claim 34 wherein said at least one sensor is mechanically coupled to at least one of said RF electrodes.

36. The system according to claim 29 wherein said system comprises a hand held unit including one or more of said RF electrodes and said heating unit and a base unit connected to said hand held unit, said base unit includes at least said RF generating unit.

37. The system according to claim 36 wherein said base unit also includes a processor/controller unit for controlling the operation of said RF generating unit.

38. The system according to claim 37 wherein said base unit also includes an impedance determining unit for determining the value of the electrical impedance between said RF electrodes, said impedance determining unit is connected to said processor/controller unit and to said RF electrodes, said processor/controller unit is configured for terminating the application of RF currents to the skin when the value of the impedance between said RF electrodes exceeds a threshold impedance value.

39. The system according to claim 37 wherein said base unit also includes a speed determining unit connected to one or more sensors included in said hand held unit for determining the velocity of movement of said hand held unit along the skin, wherein said heating unit is a movable heating unit and wherein said processor/controller unit is configured for terminating the heating of said heating unit if the value of said velocity of movement is less then a threshold value.

40. The device according to claim 28 wherein said RF current generating unit has a nominal RF power rating in the range of 1-20 watt.

41. The device according to claim 28 wherein said RF current generating unit is configured for providing RF energy to said skin in an energy delivery mode selected from pulsed RF, continuous wave RF and quasi-continuous wave RF.

42. The device according to claim 28 wherein said RF current generating unit is configured for providing RF voltages having an RF frequency range of 200 KHz-100 MHz.

43. A method for hair removal, the method comprising the steps of:
applying to the skin RF electromagnetic energy; and
applying heat from a heating element to at least one portion of at least one hair of said skin.

44. The method according to claim 43 wherein the step of applying heat from a heating element comprises heating said at least one portion sufficiently to induce a tissue coagulation wave propagating along at least a portion of the tissues surrounding said at least one hair.

45. The method according to claim 43 wherein the step of applying heat from a heating element also comprises heating said at least one portion sufficiently to cut said at least one hair.

46. The method according to claim 43 wherein the step of applying heat from a heating element also comprises heating said at least one portion sufficiently to ignite said at least one hair.

47. The method according to claim 43 also comprising the steps of moving said heating element along the skin, determining the velocity of movement of said heating element relative to said skin, and terminating the heating of said heating element if the value of said velocity of movement is smaller then a threshold value.

48. The method according to claim 47 also comprising the step of initiating the heating of said heating element if the value of said velocity of movement is larger than said threshold value.

49. The method according to claim 43 also comprising the step of moving said heating element along the skin, determining the velocity of movement of said heating element relative to said skin, and increasing the distance between said heating element and said skin if the value of said velocity of movement along said skin is smaller then a threshold value.

50. The method according to claim 49 also comprising the step of decreasing the distance between said heating element and said skin if the value of said velocity of movement along said skin is larger than said threshold value.

51. The method according to claim 43 wherein the step of applying to the skin RF electromagnetic energy comprises the step of passing RF currents through said skin by applying RF voltage to said skin.

52. The method according to claim 51 wherein said RF electromagnetic energy has a nominal RF power in the range of 1-20 watt.

53. The method according to claim 51 wherein said RF electromagnetic energy is applied in an energy delivery mode selected from pulsed RF, continuous wave RF and quasi-continuous wave RF.

54. The method according to claim 51 wherein said RF electromagnetic energy has an RF frequency range of 200 KHz-100 MHz.

55. The method according to claim 51 wherein said RF voltage is a pulsed RF voltage having a pulse repetition rate in the range of 100 Hz-25 KHz and an RF pulse duration in the range of 0.01-2 milliseconds.

56. The method according to claim 43 wherein the step of applying to the skin RF electromagnetic energy also comprises the steps of:
determining the value of the impedance between at least two RF electrodes used for applying said RF electromagnetic energy to said skin; and
terminating the applying of RF electromagnetic energy to said skin if the value of said impedance exceeds a threshold value.

57. The method according to claim 56 wherein the step of applying to the skin RF electromagnetic energy also comprises the step of initiating the applying of RF electromagnetic energy to said skin if the value of said impedance is smaller than said threshold value.

58. A method for hair removal, the method comprising the steps of:
subjecting the skin to RF electromagnetic radiation; and
applying heat to a portion of at least one hair of said skin to reduce the resistance of at least a portion of tissue adjacent to said at least one hair to flow of RF currents therethrough to form in skin tissues adjacent said at least one hair a moving coagulation zone advancing in said skin tissues to at least partially coagulate a portion of said skin tissues.

59. A method for hair removal, the method comprising the steps of:
applying to the skin RF electromagnetic energy insufficient to cause tissue coagulation of said skin by itself; and
applying heat to at least one portion of at least one hair of said skin, said applying ignites said at least one hair and produces an RF current induced tissue coagulation wave propagating along at least a portion of the tissues surrounding said at least one hair.
